(19) United States
(12) Patent Application Publication
(10) Pub. No.: US 2013/0261533 A1
(43) Pub. Date: Oct. 3, 2013

(54) SINGLE OPERATOR ANESTHESIA AND DRUG DELIVERY SYSTEM

(71) Applicant: Matthew W. Norkunas, Denver, CO (US)

(72) Inventor: Matthew W. Norkunas, Denver, CO (US)

(21) Appl. No.: 13/825,696

(22) PCT Filed: Sep. 23, 2011
(86) PCT No.: PCT/US11/52895

§ 371 Date: Jun. 24, 2013

Related U.S. Application Data
(60) Provisional application No. 61/368,056, filed on Jul. 27, 2010.

(51) Int. Cl. A61M 19/00

(52) U.S. Cl. CPC A61M 19/00
USPC 604/21

(57) ABSTRACT

The single operator anesthesia and/or drug delivery system disclosed herein incorporates a variable-output nerve stimulator and an aspirating syringe pump, both of which are controlled via a needle with controls for stimulation and aspiration/injection on the needle itself. Pre-set injection pressures can be monitored and regulated via the syringe pump. Further pressures can be objectively limited by the syringe pump. With this system, the single operator can with sterile conditions, incorporate ultrasound, nerve stimulation, and landmark palpation while simultaneously performing the block without any help from another caregiver/operator. This system can easily store or translate to the electronic medical record the specifics of the block, including for example pressures and stimulation level). The present invention further provides a means to remove all direct human elements of a nerve block and perform a nerve injection robotically or from a remote location.

### CROSS-REFERENCE TO RELATED APPLICATIONS

This application claims priority to the U.S. application 61/386,056 filed on Sep. 24, 2010, the contents of which are incorporated in its entirety.

### FIELD OF THE INVENTION

The present invention relates to anesthesia and drug delivery related products. Specifically, the present invention relates to a product designed to allow a single person to provide a regional pain block, alternatively in conjunction with accepted modalities associated with a regional pain block, in a sterile fashion.

### BACKGROUND OF THE INVENTION

The goal of regional anesthesia (aka “nerve block” or “block”) is simple: to make a specific region of the body insensate so that a person can have a procedure done on that portion of the body without any pain. If the region of the body to be operated on cannot be rendered fully insensate by regional anesthesia, a block can be used to significantly reduce the pain experienced by a patient. Essentially, local anesthesia can be deposited around any nerves in the body to make the body parts associated with those nerves numb. When blocking large nerves at this level, the term “numb” refers to a lack of sensation, motor function, and any other function carried in these large fibers with mixed functions contained within. The goal of a nerve block is to make a limb or body part feel “asleep” (i.e. how a dentist numbs a tooth). The current system for delivering that local anesthetic is fairly complex and cumbersome, requiring at least two people and multiple components.

The main components of available systems used to provide regional anesthetics are: a hollow metal needle, a nerve stimulator, at least one syringe, and an ultrasound. Each is discussed below.

The first aforementioned component is a hollow metal needle through which local anesthesia can be injected onto/around target nerves. Generally these needles are 50-150 mm long and 21 or 22 gauge. They are blunt-tipped and are often insulated on the outside of the needle except for the tip, to allow for accurate placement of the needle tip via a nerve stimulator. There is sometimes an addition to a nerve block which is called a “continuous catheter.” Essentially, instead of just injecting local anesthesia through the needle and removing the needle completely, the needle can be used to introduce a small plastic catheter which stays inside the patient right next to the nerve after the needle is removed. This catheter can be used to infuse local anesthesia around the nerve for up to a week. These catheters must be placed in a sterile fashion, failure to do so can result in infection.

A nerve stimulator is attached to the needle and sends electrical pulses down the length of the needle to the needle tip. When the needle tip is close to the target nerve, the muscles associated with that large mixed nerve fiber will twitch. This signals that the needle is close to the target nerve. As the nerve stimulator output is “dialed down” at the level of the control box, the placement of the needle tip can be focused closer and closer to the target nerve. If a muscle twitch is seen with an output of 0.5 mA or less, it is widely accepted that injection of the medication through the needle near the nerve will result in adequate nerve blockade.

A syringe is connected to the hollow needle via a clear plastic tubing for the purpose of delivering the local anesthetic. When the needle tip is felt to be in the appropriate location, the caregiver (generally not the needle operator), operating the syringe will aspirate gently on the syringe. This negative pressure is applied to make sure the needle tip is not in a blood vessel or in another body cavity where the needle tip should not be. If blood (needle tip in a vessel) or air/feces (needle tip in lung or bowel) comes back into the clear plastic tubing via the hollow metal needle, the needle needs to be repositioned and no medication should be injected. If the aspiration is “negative,” meaning no unexpected contents are seen in the tubing and a vacuum can be felt in the tubing by the syringe operator, medication can be injected. Emerging literature is showing that higher injection pressures (above 40 mmHg at the needle tip) may be associated with a higher incidence of nerve injury from such nerve blocks. It is very difficult for the syringe operator to objectively determine the pressure of the injectate. It is also presently not generally possible to provide at a later date proof or evidence of the injectate pressure that was administered.

An ultrasound can be used during a regional block to visualize the tissues, nerves, needle and spread of local anesthesia in real time. This modality has made regional anesthesia more reliable, faster, and (potentially) safer than in the past. The ultrasound is generally held by the needle operator in the hand opposite the needle. Ultrasound controls are generally provided on the ultrasound unit that relate for example to imaging and depth.

In essence, a regional nerve block is a complex interplay of two or more individuals using a needle, an ultrasound, a nerve stimulator, and syringes to inject local anesthetics directly onto targeted nerves at low pressures. There are many pitfalls within this interplay that can result in significant patient harm, including intravascular injection of local anesthetics, puncture of lung or bowel, and high pressure injections directly into nerves. Safe, reproducible coordination of all the modalities while being considerate of the potential complications can be very difficult for two operators. Furthermore, it is imperative that the needle, the tubing, the syringes and any stimulator wires that come in contact with the patient be packaged sterilely and used only once as reuse of any portion of the components coming in contact with the patient can result in a catastrophic infection.

There are several needs related to current nerve block technology. For example and as mentioned, the technology as currently practiced requires at least two caregiver/operators. Assistance is needed to aspirate the syringes, inject the local anesthesia, help run the nerve stimulator, operating the ultrasound, and the like. Requiring more than one caregiver/operator increases the costs related to the care.

Further, emerging literature in anesthesia is associating nerve injury (and ensuing legal actions) with high-pressure injections. Available pressure monitors are generally very subjective, not terribly reliable and for these and other reasons are often simply not used. For example Hadzic provides a “turkey-timer” like monitor that is added in-line between the tubing and the needle. The center of the monitor pops out as the caregiver injects, and it sticks out further with higher pressures. There are strips painted on the pop-out part that give a range of what the pressures may be, but the monitor is not standardized for specific syringes/needle lengths or gauges/tubing compliances or lengths, etc. See for example U.S. Pat. No. 5,830,151; U.S. Pat. No. 7,689,292; and U.S. Pat. No. 7,727,224.

Generally, the caregiver/operator performing the block holds an ultrasound probe or palpates an artery w/ one hand and holds the aspiration/delivery needle with the other. Another person generally injects the medications and/or alters the output of the nerve stimulator. If it feels “tight” (i.e. high injection pressure), the caregiver might not inject, or if they are not experienced with injecting they may just inject even if it is high pressure. This is a real problem. The pressure monitor is the sensitivity of the hands of the syringe operator and is thus only as precise as the syringe operator's experience.

In addition, there are often different sizes of syringes used for injecting the medication. Each syringe size (60 cc, 20 cc 10 cc) requires a different amount of energy added at the level of the plunger to inject the medication, thus each syringe “feels” different to the syringe operator, even if the pressures at the tip of the needle are the same.

Because the needle and its attached tubing and stimulator wires come sterile, the hand of the needle operator is generally sterile. That needle operator also generally has the ultrasound probe in the other hand and that probe generally has a sterile cover on it. The tubing and the wires that hang off of the needle basically plug into the stimulator and the syringes. However, the second caregiver/operator is generally not sterile. The second operator controls the syringes and the stimulator itself. See for example prior art illustration FIG. 7. The stimulator base is re-usable and is not in the sterile field. The syringes will be thrown away after they're used. The second operator also helps manipulate the ultrasound controls, which are similar to a laptop computer keyboard in most configurations. Such controls are difficult to sterilize.

As mentioned, the needle/ultrasound probe operator is sterile (this operator is breaking the patient's skin which puts the patient at risk for infection). Sterility is particularly important if the needle operator is placing a continuous catheter. Having a second non-sterile caregiver present increases the opportunity for contamination of the patient increasing infection risk.

It can be difficult and time consuming to enlist a second caregiver/operator to assist with a nerve block. Furthermore, if that person is inexperienced, the injection pressure can be extremely an inappropriately high and unregulated by the first caregiver/operator who is actually performing the block.

What is needed is a means to allow a single person to provide a sterile-environment nerve block with improved control, consistency and reproducibility.

### SUMMARY OF THE INVENTION

The claimed invention provides means to allow a single person to provide a sterile-environment nerve block with improved control, consistency and reproducibility. The present invention addresses the potential risk of nerve injury due to high-pressure injection by providing a means to directly measure and regulate the pressure of injectate and to choose volumes of injectate to be injected.

It is an object of the invention to provide a single operator anesthesia and/or drug delivery system (SORADS).

It is an object of the invention to provide a means to directly measure and regulate the pressure of injectate, and choose volumes of injectate to be injected.

It is a further object of the invention to reduce time to block.

It is a further object of the invention to provide a drug delivery system administrable by a single caregiver/operator.

It is a further object of the invention to provide a means to objectively measure injectate pressures.

It is an object of the invention to provide a means to document the injectate pressure administered.

It is a further object of the invention to provide a means to objectively measure and document injectate pressures.

It is a further object of the invention to provide a means to transmit all information to an electronic medical record.

It is a further object of this invention to provide the ability for a single operator to administer a peripheral/regional nerve block in a sterile fashion using all accepted/billable/documented modalities.

It is a further object of this invention to provide the ability for a single operator to administer a peripheral/regional nerve block in a sterile fashion in conjunction with ultrasound.

It is a further object of this invention to allow a single operator to provide either a ‘single shot’ or place a continuous nerve block catheter in a sterile fashion.

It is a further object of this invention to have precise, mechanized measuring of the pressures of the injectate as it leaves the needle, to minimize high-pressure injections.

It is a further object of this invention to provide a single button which serves as a gate, wherein aspiration and injection is controlled at the level of the syringe pump on the base unit.

It is a further object of this invention to provide a two button system, having separate aspiration and injection controls on the base unit.

It is a further object of this invention to provide a single button system, where the single injection button is on the housing of the needle that acts as a toggle to control separate aspiration and injection controls on the base unit.

It is a further object of this invention to have an aspirating syringe pump controlled by the block needle.

It is a further object of this invention to have a nerve stimulator, the output of which can be controlled at the level of the block needle via a dimmer switch in the housing of the needle itself or in close proximity to the tubing of the present invention.

It is a further object of this invention to have a needle and/or tubing with a housing that has control(s) allowing for precise control of injectate flow, pressure, and volume as well as allowing for precise control of milliamperage distributed to the needle tip via a variable output/resistance “dimmer switch”.

It is a further object of this invention to be compatible with robotic/remote nerve block technology where the operator need not be present to perform the block.

It is an object of the invention to provide a single operator anesthesia and/or drug delivery system that can communicate with ultrasound and/or other modalities by way of blue tooth or other wireless technology, where such communication can be used to control the modality and/or to record data from the modality.

These and other objects are achieved in the present invention, which enables the performance of a sterile, one-handed injection of a medication. A device in accordance with claimed invention enables, in connection with the one handed injection, aspiration/injection, variable output nerve stimulation, objective injection pressures, and the ability to communicate, via Bluetooth or other wireless communication means, with the various controls of an ultrasound device, including controls related to imaging, depth of beam alterations, and the like.

There has thus been outlined, rather broadly, exemplary features of the invention in order that the detailed description thereof that follows may be better understood, and in order that the present contribution to the art may be better appreciated. There are, of course, additional features of the invention that will be described further hereinafter.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that equivalent constructions insofar as they do not depart from the spirit and scope of the present invention, are included in the present invention.

For a better understanding of the invention, its operating advantages and the specific objects attained by its uses, reference should be had to the accompanying drawings and descriptive matter which illustrate preferred embodiments of the invention.

### BRIEF DESCRIPTION OF THE DRAWINGS AND THE FIGURES

FIG. 1 is a diagrammatic illustration of a single operator anesthesia delivery system.

FIG. 2 illustrates a single button embodiment where the button is not depressed.

FIG. 3 illustrates a single button embodiment where the button is depressed during an aspiration/injection cycle.

FIG. 4 shows a diagram of an exemplary single operator anesthesia delivery system.

FIG. 5 shows a diagram of a robotic anesthesia delivery system.

FIG. 6 illustrates an embodiment of the present invention.

FIG. 7 illustrates a prior art system requiring at least two caregivers/operators.

FIG. 8 illustrates an exemplary system of the present invention system requiring only one caregiver/operator.

FIG. 9 illustrates hydraulic and pneumatic circuit diagram of an exemplary prototype.

FIG. 10 illustrates electrical circuit diagram of an exemplary prototype.

FIG. 11 illustrates a 3 position fill and bleed valve.

FIG. 12 illustrates hydraulic and pneumatic circuit diagram with three-position custom valve.

FIG. 13 illustrates electrical circuit for a dimmer/interval switch.

FIG. 14 illustrates alternate views of an exemplary device casing

FIG. 15 illustrates exemplary engineering drawings for a device casing.

FIG. 16 illustrates exemplary engineering drawings for a device casing cap.

### DETAILED DESCRIPTION OF THE INVENTION

### Overview

The claimed invention gives a single caregiver/operator access to and control of procedures and operations which, in the prior art generally requires a second operator/caregiver. Multiple embodiments of the present invention are provided herein. An element in common with these embodiments is that each provides a means by which a single caregiver can provide a sterile “single shot” block as well as a sterile placement of a nerve block catheter utilizing all of the current technologies associated with nerve blocks. As disclosed herein this single operator administrable anesthesia and drug delivery system enables controlled aspiration/injection, objective measurement and documentation of injection pressures, and the ability to communicate via Bluetooth or other wireless methods, with the various controls of an ultrasound device, including controls related to imaging, depth of beam alterations, and the like. Thus for example, the SORADS of the present invention can communicate with ultrasound and/or other modalities by way of blue tooth or other wireless technology, where such communication can be used to control the modality and/or to record data from the modality.

Three general exemplary embodiments are provided herein. One is a single button embodiment, wherein the single button on the needle housing serves as a gate, and aspiration and injection is controlled at the level of the syringe pump. A second, is a two button system, having separate aspiration and injection controls on the base unit as well as on the needle housing. A third, is a single button system, where the single button on the needle housing serves as a toggle control for the separate aspiration and injection controls at the base unit.

In each embodiment provided herein and in additional embodiments as would be understood by one of ordinary skill in the art, all related information including but not limited to mA, injection pressures, medical images such as ultrasound images, (including for example, an image of the needle while the block is being performed), volume of injectate, stimulation levels achieved with the stimulator, time necessary to complete the block and the like can be recorded and saved to a computer readable medium such as a USB memory device, DVD, zip drive, CD or directly entered into the electronic medical record where all patient data can be entered or scanned into the machine. For example, such information can be saved to the base unit.

In the illustrative embodiments provided herein and in other alternative embodiments, additional pressure control can be provided to ensure that the injection or aspiration pressure is not too high. For example, check valves or pressure limiting valves in the handle that stop flow at high pressures can be incorporated.

### Figures

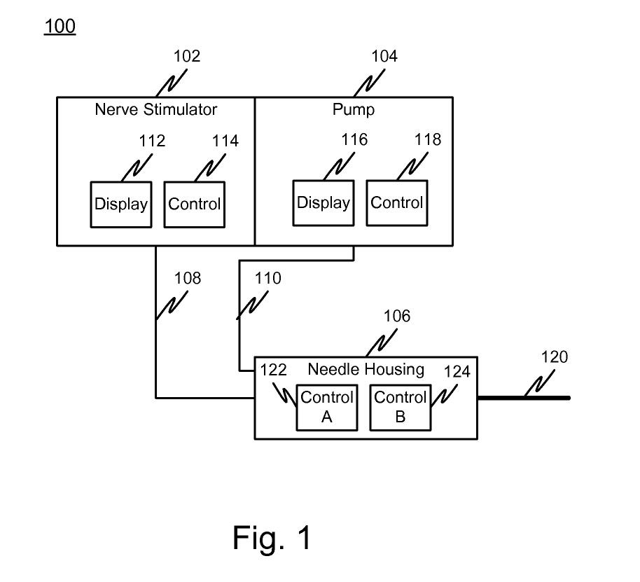

FIG. 1 is a diagrammatic illustration of a single operator anesthesia and drug delivery system. In particular, the system 100 includes a nerve stimulator 102, a pump 104 and a needle housing 106. The nerve stimulator 102 is coupled to the needle housing 106 via an electrical link 108. The pump 104 is coupled to the needle housing 106 via tubing 110. The nerve stimulator 102 includes a display 112 and a control 114. The pump 104 includes a display 116 and a control 118. The needle housing 106 includes a needle 120, a first control 122 and a second control 124. The needle 120 can be a 22 gauge blunt insulated needle, for example. A control C can be included, wherein Control C is integrated or is snap-on/attachable with/to Bluetooth ultrasound control.

In operation, the nerve stimulator delivers an electrical nerve stimulation signal to the needle housing 106 via the link 108. The intensity of the nerve stimulation signal can be controlled by the control 114 and the intensity setting can be displayed on display 112. Also, control A 122 on the needle housing can be used to regulate the intensity of the nerve stimulation signal. The nerve stimulator control 122 and 114 can be for example a rotary or sliding control (e.g., a potentiometer), or the like.

The pump 104 provides an aspiration and injection function to the needle 120, via the tubing 110 and the needle housing 106. The pressure of the pump can be controlled via the control 118 or control B 124 on the needle housing 106. The pressure setting can be displayed on the display 116. In one embodiment, control B 124 includes limits/settings options for the system

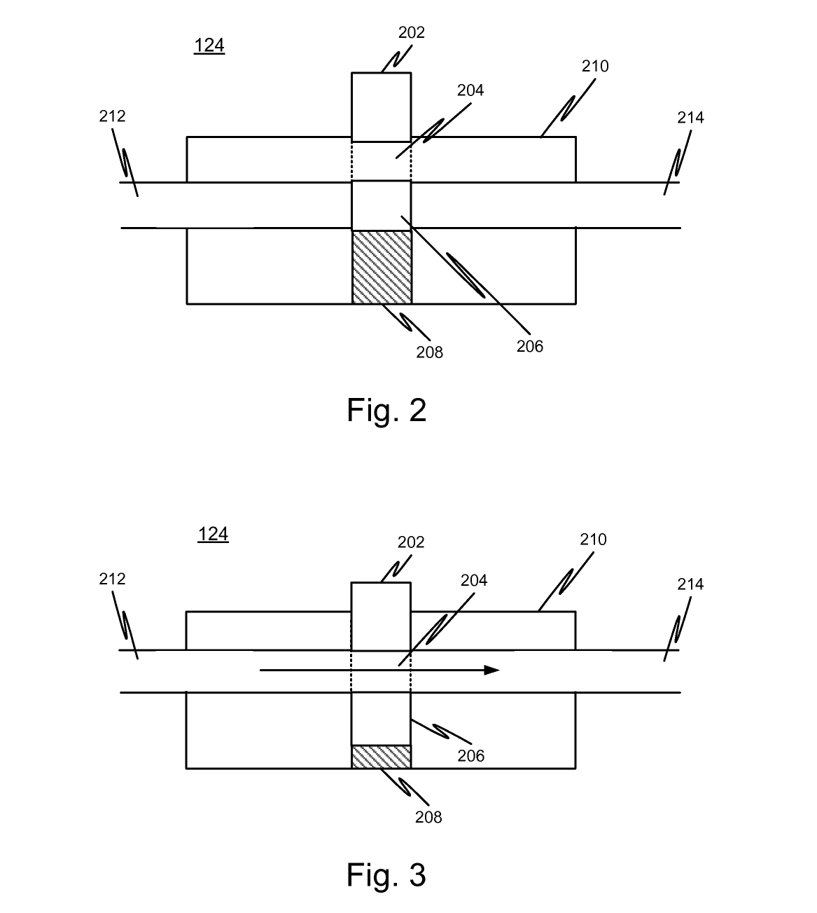

When control B 124 is depressed, the pump can aspirate the needle for a predetermined period of time (for example, 1 second). Then, the pump can begin to pump anesthetic through the tubing 110 according to the pressure setting. The pump control 124 can be a valve-type control, as shown in FIG. 2 and FIG. 3. The pump control 124 can also be an electrical control coupled to an electrically actuated valve or the like. The control 118 can be a rotary or linear control. Alternatively, the controls and displays could be merged into a touch screen device on the pump 104 that includes control and display capabilities.

FIG. 2 and FIG. 3 show an exemplary pump control valve such as control 124 disposed within the needle housing 106. At a closed position, shown in FIG. 2, the button 202 and opening portion 204 have been raised by force of a spring 208 within a control body 210 such that opening 212 is blocked by solid member 206 from being in connection with opening 214. At an open position, shown in FIG. 3, button 202 has been depressed, solid member and opening 204 have been moved such that opening 212 is in communication with opening 214 and liquid can flow between openings 212 and 214.

The medication injection can be accomplished at a pre-set rate and provided with a pre-set pressure limit. All rates and limits are set at the level of the base unit. In one embodiment this is a variable pressure. As illustrated by FIG. 2 and FIG. 3, when it is desired that the injection be discontinued, the finger is taken off the button, the alignment of the button portion is out of line with the flow of the injectate and there is no more flow through the needle. If more injection is desired, the cycle starts again by depressing the button.

As illustrated by FIG. 2 and FIG. 3, when the button on the needle having a first diameter, lines up with the tubing of the same diameter (due to being pressed by the caregiver/operator) a pressure change in the line at the level of the needle housing provides the signal to the pump causing aspiration for a pre-determined amount of time. The first diameter is the “gap” in the button as well as the diameter of the tubing and the needle itself.

In practice, with the claimed invention, a caregiver/operator uses one hand to hold an ultrasound or locate a landmark (artery) while the other hand holds the needle. The needle can be placed into the desired area. The needle housing has a “dimmer switch” that can be slid up and down during placement to allow variable control of the delivery of current to the needle tip for nerve stimulation. In one embodiment a range of 2.0-0.2 mA is provided. In a further embodiment, the switch provides small “stops” to offer support to the finger using the switch and to provide feedback as to setting. The switch alters the output of the nerve stimulator which is attached to the needle via a wire from the large base-unit. This output can be reflected on a mA readout on the stimulator portion of the base-unit. An injection button, which controls the delivery of injectate, is also provided. When an injection is desired, the injection button is depressed.

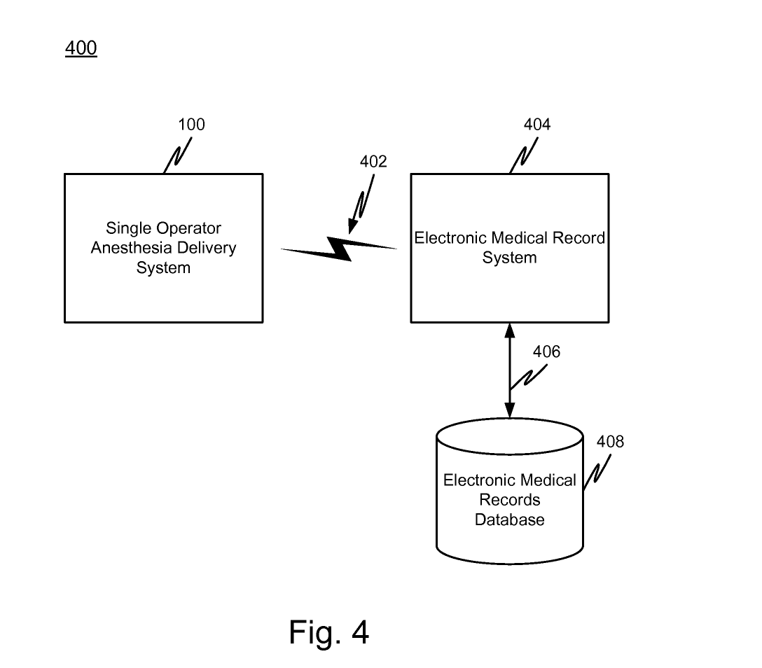

FIG. 4 shows a diagram of an exemplary single operator anesthesia delivery system coupled to an electronic medical record system. In particular, a health care system 400 includes a single operator anesthesia delivery system 100 coupled to an electronic medical record system 404 via a link 402. The electronic medical record system 404 includes a medical record database server 408 coupled to the electronic medical record system 404 via a link 406. Links 402 and 406 can be a wired or wireless link, such as a LAN, WAN, Ethernet, Internet, WiFi, Bluetooth, or the like.

In operation, data captured during an anesthesia procedure by the single operator anesthesia delivery system can be transmitted via the link 402 to the electronic medical record system and stored in the database 408 in a record associated with the patient being anesthetized. The data can includes the pump pressure and nerve stimulation settings, the amount of energy delivered via the nerve stimulator, the amount of anesthetic delivered, readings from the controls, ultrasound images, and any other information associated with the anesthesia procedure. In one contemplated embodiment the SORAD 100 provides local storage of this data.

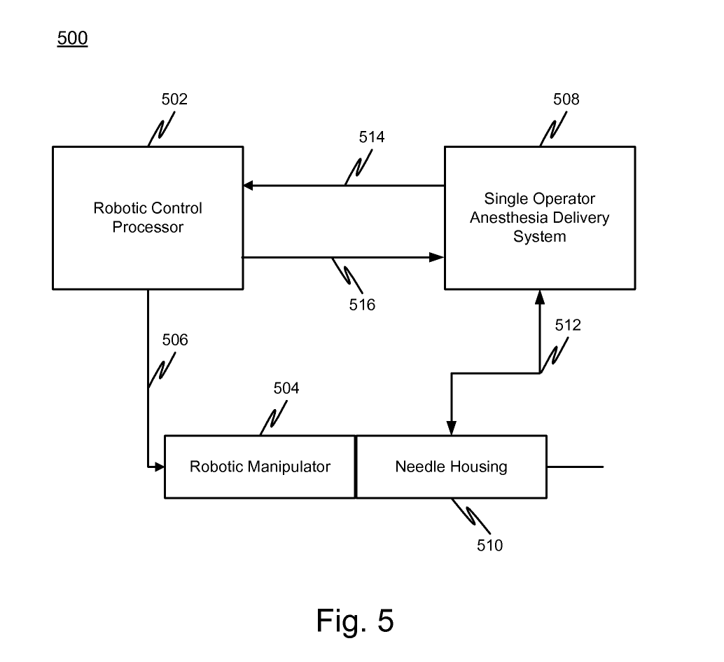

FIG. 5 shows a diagram of a contemplated robotic anesthesia delivery system including a single operator anesthesia delivery system in accordance with the present invention. In particular, a robotic anesthesia system 500 includes a robotic control processor 502 coupled to a robotic manipulator 504 via a control/feedback link 506. The robotic system 500 also includes a single operator anesthesia delivery system 508 coupled to a needle housing 510 via an electrical/tubing link 512. In this embodiment the robot “controls” the SORADS system with it's 2 “hands” in place of the current operator's hands.

In operation, the robotic manipulator 504 holds (or is connected to) the needle housing 510. The robotic manipulator 504 positions the needle housing 510 to perform an anesthesia procedure. The robotic control processor 502 receives signals from the anesthesia delivery system 508 via link 514 and provides control input to the anesthesia delivery system 508 via control link 516. Thus, the robotic control processor 502 can signal the manipulator 504 to move so as to perform the anesthesia procedure while monitoring the signals and providing control to the anesthesia delivery system 508. Alternative embodiments do not require link 514 or control link 516. Here, the robot controls the SORADS at the level of the needle housing, and direct contact between the robot and the base unit is not required. The robot is controlled remotely by a human. The present invention therefore provides a means towards eliminating all direct human operation of a nerve block. Use of robotic arms to drive the ultrasound and hold the needle while the block is performed from a remote location through the use of this system is contemplated herein, as illustrated by FIG. 5.

The robotic anesthesia system can include a known or later developed surgery robot integrated with the anesthesia delivery system. For example, the da Vinci Surgical System (manufactured by Intuitive Surgical, Inc., of Sunnyvale, Calif.) could be integrated with an embodiment of the anesthesia delivery system to provide a robotic anesthesia system. A robotic anesthesia system may be semi-automatic or fully automatic.

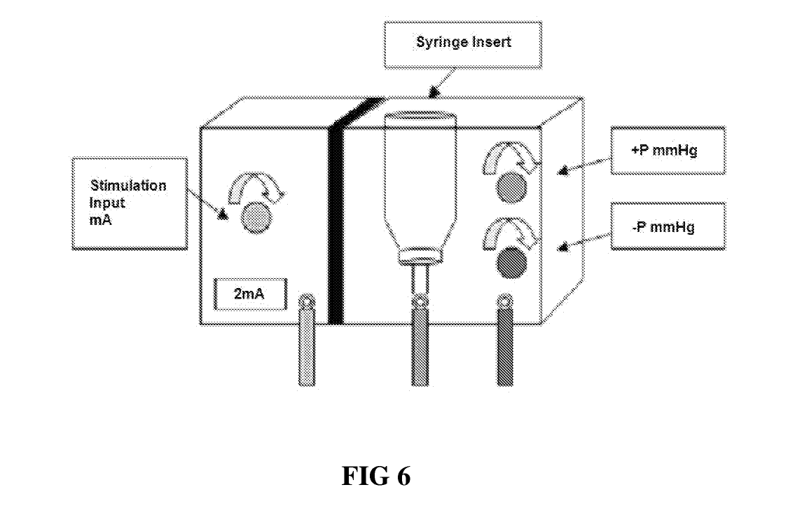

FIG. 6 further illustrates a base unit of a two button embodiment of the present invention. As shown, this embodiment includes two separate aspirating/injection tubings in the base unit.

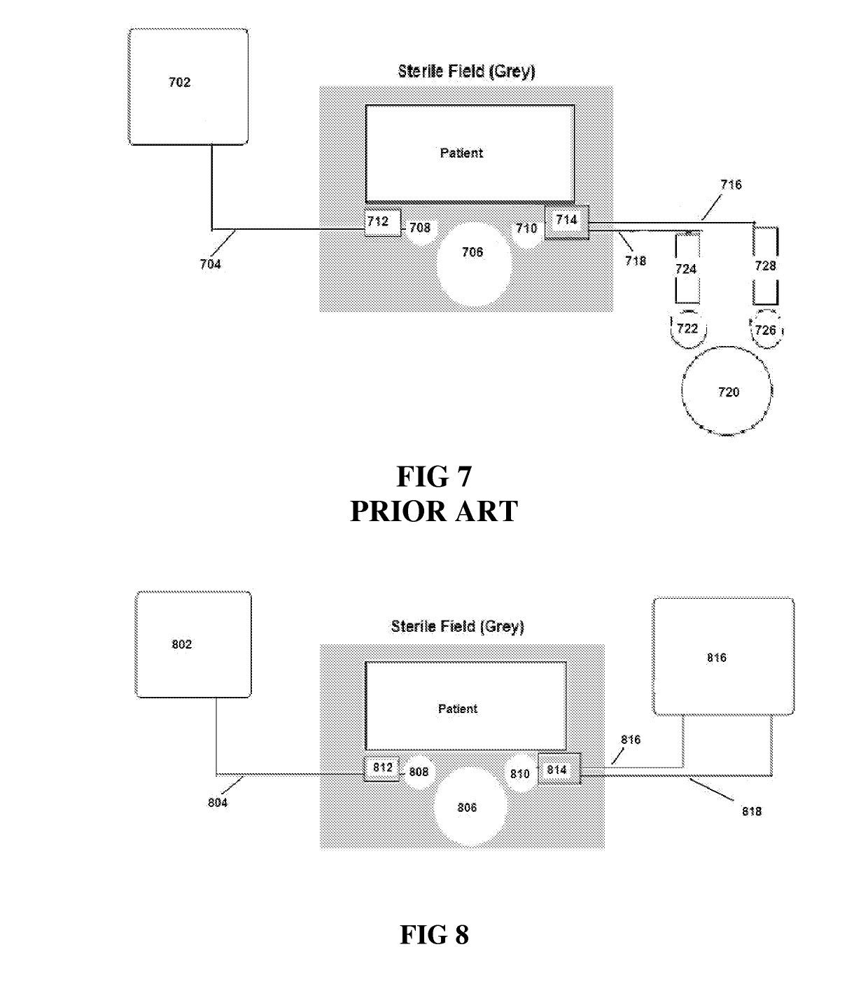

FIG. 7 illustrates a prior art system requiring at least two caregivers/operators and showing ultrasound screen and unit 702, ultrasound probe cable 704, first caregiver 706, first caregiver hand one 708 operating probe 712, first caregiver hand two 710 operating block needle 714, injectate tubing 716, wiring 718, second caregiver 720, second caregiver hand one 722 operating nerve stimulator 724, and second caregiver hand two 726 operating syringe 728.

In contradistinction, FIG. 8 illustrates an embodiment of the present invention requiring only one caregiver/operator, showing ultrasound screen and unit 802, ultrasound probe cable 804, sole caregiver 806, sole caregiver hand one 808 operating probe 812, sole caregiver hand two 810 operating block needle with the SORADS base unit 814 providing stimulation control and injectate control. Also shown are wire to stimulation 816 and injectate tubing 818.

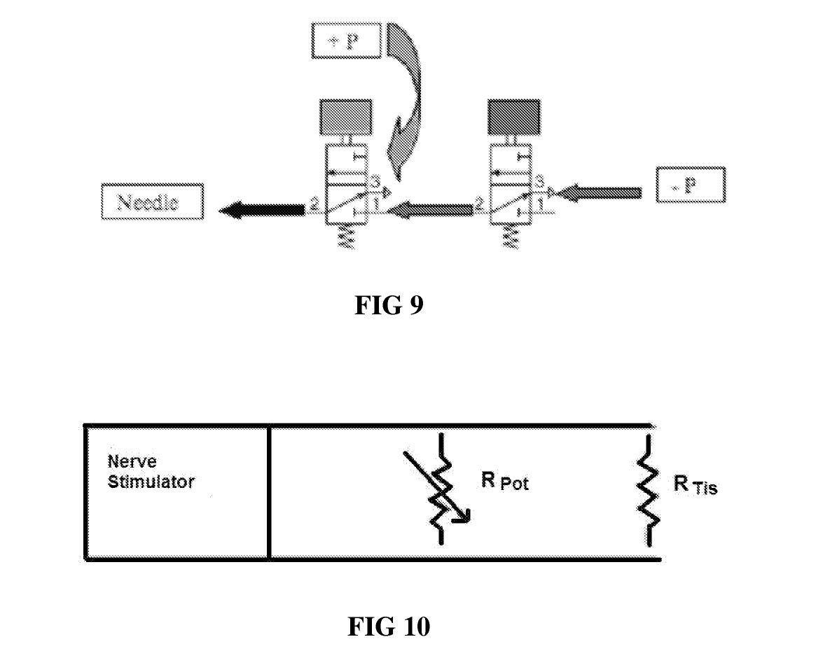

FIG. 9 illustrates a hydraulic and pneumatic circuit diagram for an exemplary two button device. In this embodiment a valve was used having three ports: “Normally Open” (NO), “Normally Closed” (NC), and OUT. When the button is not depressed, the N.O. port is open and allows flow to the OUT port. When the button is depressed, the N.O. port is blocked and the N.C. is open and flows to the OUT port. In this exemplary embodiment, the valve was small (0.95 inches long and 0.3125 inch hexagonal diameter). In this exemplary embodiment, one button (red) controls aspirating and the other button (green) controls injecting. When neither button is pressed, the system is “off.” In FIG. 9 “+P” represents a positive pressure source (anesthetic) and “−P” represents a negative pressure source (vacuum). Alternatively, an embodiment of the single button system can include a mechanical arm that will pull back on the syringe and push forward on that same syringe when a negative aspiration is sensed.

FIG. 10 illustrates a circuit diagram providing a current divider circuit with a potentiometer to be housed in the handle. In this embodiment the stimulator can be set at a constant value and controls for attenuation of the current are provided in the handle. Here, RTIS represents the resistance of the tissue where the needle is inserting. In certain embodiments this is estimated as 1 kΩ. The RPOT represents the resistance of the potentiometer which can be varied between 0 and 10 kΩ based on the location of the wheel. When RPOT=10 kΩ, that arm of the circuit will have such a high resistance that all input current will flow through the tissue. When RPOT=0 kΩ, that arm of the circuit will essentially act as a wire so it will draw all the current down that arm of the circuit and the tissue will experience no current stimulation. As the RPOT decreases from 10 to 0 kΩ, the potentiometer arm of the circuit will draw current away from the tissue until it experiences zero or very little current. This circuit response achieves the desired functionality, allowing the anesthesiologist to begin stimulating with a large amount of current and gradually attenuate the current down to nearly zero as the needle is placed close to the nerve.

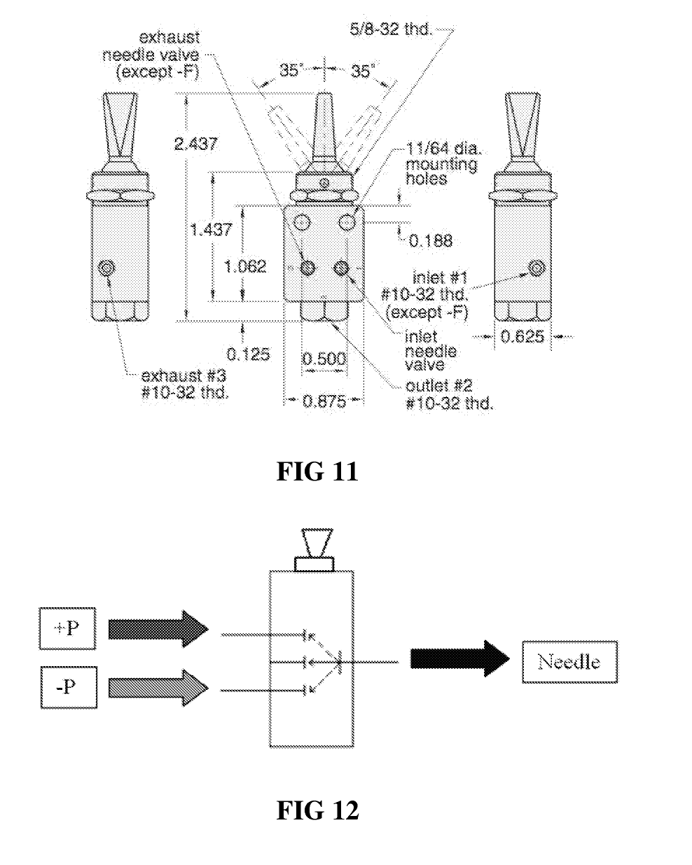

FIG. 11 illustrates a 3 Position Fill & Bleed Valve. As mentioned prior, in one embodiment of a one button design of the present invention, a mechanical arm is pulled forward and backward on the syringe. Alternative embodiments incorporating a one button design are further contemplated herein. For example, in one contemplated embodiment a 3 Position Fill & Bleed Valve such as illustrated in FIG. 11 is miniaturized. This type of valve provides three settings. When the toggle is upright all ports are blocked. If the toggle is pressed one way, one input opens to the outlet, and if the toggle is pressed the other way, the second input opens to the common outlet.

FIG. 12 illustrates a sample hydraulic and pneumatic circuit diagram that could be used with this valve. In a further contemplated embodiment, in addition to miniaturizing the 3 Position Fill & Bleed Valve valve, the toggle switch is replaced a rocker switch where there can be three positions (forward=injecting, normal=off, and back=aspirating). In a further alternative embodiment, a D-pad similar to ones on a video game controller is used.

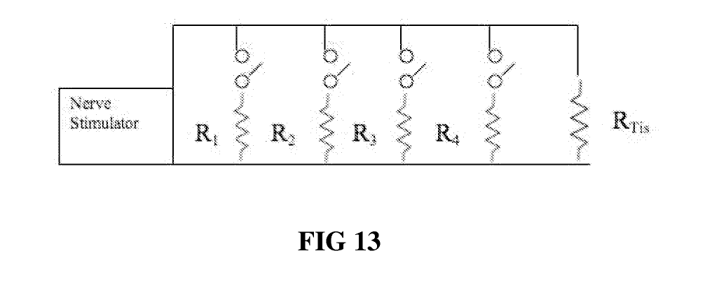

FIG. 13 illustrates the potential switch circuit where the resistance values R1-R4 vary based on the desired current. Each position on the dimmer switch would close the corresponding switch to activate the desired current divider. This embodiment can provide the Caregiver with feedback on the stimulation control. For example, a display on the handle can show the amount of current flowing through the tissue. Alternatively, the dimmer wheel can be replaced with a switch that has set positions at 0.5 mA intervals. This could be achieved by replacing the potentiometer with a circuit that has switches to activate different branches of the current divider based on the desired current.

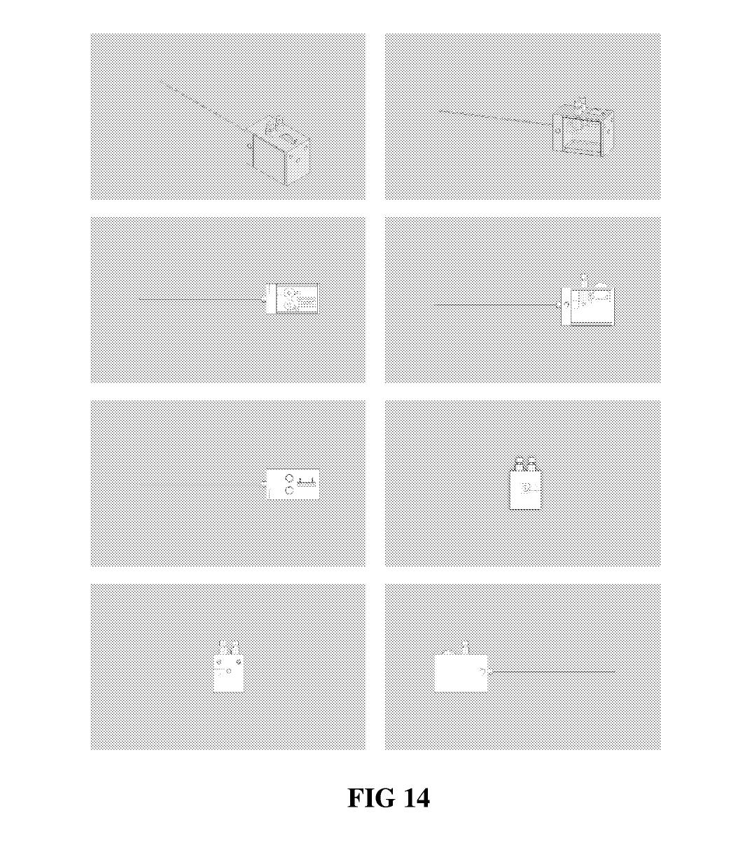

FIG. 14 provides alternative viewings of an exemplary two button system SORADS device allowing aspiration, injection and stimulation to be controlled with one hand.

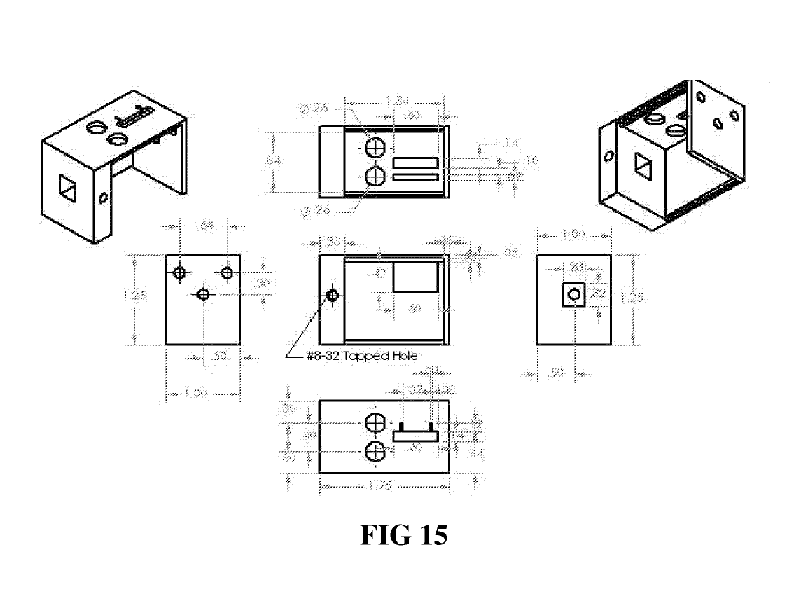

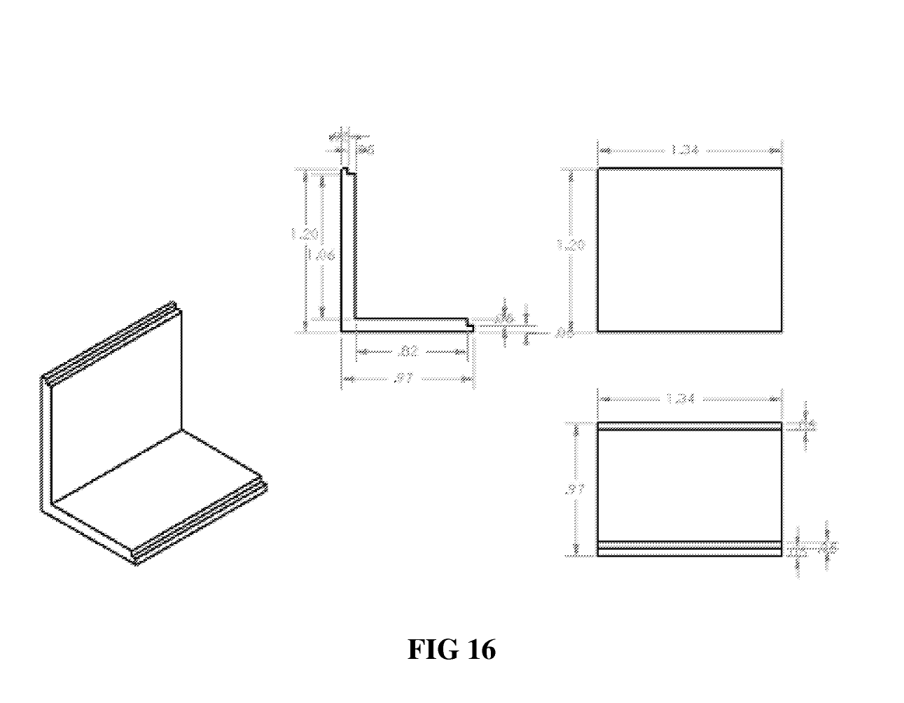

FIG. 15 and FIG. 16 provide engineering drawings of this exemplary (two button) device. As illustrated this device is assembled and includes hydraulic, pneumatic, and electrical circuits a device casing, a device casing cap, a wheel potentiometer, a needle, and polyurethane tubing. In this particular embodiment, the SORADS device is contained in a 1.75″ long×1″ wide×1.25″ tall casing. The device receives the needle on the front panel. The needle used in this embodiment is a B Braun Stimuplex A Insulated Needle; however, alternative needles are contemplated as would be know by one of ordinary skill in the art. The needle's rectangular base slides into the front of the casing and is secured using a securing means such as a screw and screw hole on the side of the casing. The needle receives two inputs from the device—one from the pneumatic/hydraulic circuit and one from the electrical circuit. 1/16″ ID Tubing connects the needle to the pneumatic/hydraulic circuit. Again, alternatives are known by one of ordinary skill in the art and these examples are provided for exemplary purposes only. The tubing connects the needle input to the output of the “injecting” valve, which has an always open connection to the “aspirating” valve and a pushbutton-activated connection to the anesthetic. Another pushbutton valve controls the aspirating functionality. A negative pressure (vacuum) source is connected to the pushbutton-activated input of the valve and the output is connected to the always-open input of the other valve. In this way, the default setting is “off”, with both injection and aspiration functions activated by individual pushbuttons. The electrical input to the needle is controlled via the previously described current divider circuit. The needle is wired to the wheel potentiometer, which allows the user to manually vary the stimulation output at the needle from the input level (maximum) to zero (minimum). Leaving out of the back of the device are the ground wire (black), which connects to the patient, and the input wire (white). The device casing is closed with a 3D printed casing cap that fits precisely into the side and bottom opening, enclosing the valve and electrical systems inside.

### Further Exemplary Embodiments

In one embodiment the injection button is on the housing of the needle. The aspirating syringe pump recognizes the alignment of the housing button diameter with the tubing by way of the pressure change through the system. Pushing the button down basically lines up the tubing with the hole in the button. In one embodiment the button is spring-loaded. The hole in the button lines up with the tubing and the needle. The syringe pump is constantly aspirating against the blocked tubing when the button is not depressed. When the button is pressed and the holes line up, if the needle is in a blood vessel or the lung/bowel, some volume of blood/air/feces starts to fill the tubing. The syringe pump recognizes this positive aspiration and does not inject. The button must be released to start the cycle over again. If the needle tip is not moved, a positive aspiration occurs again and the pump will not reverse flow to inject because the system recognizes volume (blood, air) in the tubing again. A negative aspiration means that nothing will flow backward into the tubing over the predetermined aspiration time (0.25, 0.5, 0.75 sec), and the vacuum is recognized by the syringe pump due to the lack of volume entering the tubing during that aspiration portion of the cycle. Even if a small vessel is entered and a the volume of blood entering the tubing is not enough to be recognized by the syringe pump itself, the tubing is clear and the operator can watch the tubing for blood or air and let go of the needle to re-set the aspiration/injection cycle.

In one embodiment, the recognition of alignment is accomplished through a geometrical/size conformational change brought into the tubing system by the depressed button. The needle is aspirated for a pre-set time. In one embodiment this pre-set time is in the range of about 0.25 seconds to 0.5 seconds. The syringe pump then reverses flow direction and injects the medication.

In a further embodiment an audible “aspirating” voiceover alerts the operator as to when the aspiration portion of the procedure is occurring. An exemplary embodiment encompassing an audible “injection” voice-over is described as well. Because there is no longer a need for a second operator, when the needle operator (e.g. the caregiver) pushes the button down, the syringe pump either needs to be sensitive enough to recognize the change in the vacuum, or the wire-loop version must signal the pump to say “aspirating” for the predetermined time (0.25, 0.5, 0.75 sec). With a notification such as a beep, the flow then reverses, and the voiceover says “injecting.” This amount of injectate can also be predetermined at the level of the pump (5 mL, 7 mL, 10 mL). It is also contemplated that the base unit includes a light, such as a red light, that lights up when “aspirating”, and a second light, such as a green light, that flashes on when “injecting.” If the SORADS detects a positive aspiration, it will immediately go to red and an audible alarm/specific beep can sound. Furthermore, if pressures higher than the predetermined settings are encountered, a “high pressure” audible can be used, again with the red light switching on. In practice, the green light would only go on when “injecting” under low pressures after a negative aspiration. This information can then be provided to a subsequent caregiver/operator, billing, and the like. Such information can also be electronically collected by the SORADS or printed for a hard copy of the medical record.

Use of a Bluetooth device for control of the ultrasound is further contemplated. This allows for manipulation of the ultrasound controls to optimize the ultrasound and ultimately take a picture (or store the data/picture of the block) of the needle in the correct position. Information is passed from the ultrasound portion of the Bluetooth back to the device of the present invention. This embodiment requires a controlling portion of the Bluetooth system in- or added/snapped onto a portion of the present device, such as the handpiece itself, and a receiving modality within/plugged into the ultrasound that controls the ultrasound itself. The modality then send information to the base unit of the SORADS (pictures, etc).

In one embodiment, the device can be configured to sense and recognize the presence or absence of a vacuum when aspirating the needle. If the aspirating pump is pulling a volume back (e.g., blood and/or a significant amount of air), this is an indication that the needle is not in the desired region but is instead in a blood vessel, the lung, etc. Alternatively, if the needle is in the desired region the pump will sense a vacuum during aspiration and this would indicate that the needle is properly placed. As a means of back-up, in the preferred embodiment the operator will also be able to visually observe the tubing for the presence of blood.

In an alternative embodiment of the single button system, a loop of wire acting as a circuit is integrated into the button (half of the loop) and the housing (the other half) that lines up when the button is not pressed. In this embodiment, the wire provides a signal to the pump, indicating the circuit is complete and that the pump is not to inject. Upon pressing the button, the button half of the circuit loses contact with the housing part, sending the signal to the pump via the wire, signaling the pump to aspirate. This is basically an electrical switch. When the circuit is complete, the pump does nothing. When the circuit is broken, the pump recognizes the change and goes through an aspiration/injection cycle. A voice-over can signal that the pump is aspirating and then injecting. Letting go of the button allows the circuit to be complete again, resetting the chain of events.

In a further embodiment, the present invention provides a means for a sterile “single shot” block as well as a sterile placement of a nerve block catheter. In the single shot block embodiment, after the single shot has been administered (i.e. all of the medicine has been injected into the patient), the sterile drapes are removed and nothing is left behind in the patient other than the medicine. In the sterile placement of a nerve block catheter embodiment a catheter can be left that sticks out of the skin and stays next to the nerve that allows caregiver/operators to infuse medicine into the patient for pain relief for several days. The present invention provides a means by which both the sterile “single shot” block as well as a sterile placement of a nerve block catheter can be provided by a single caregiver/operator.

As discussed, the handpiece of the claimed invention could have a single button that controls both aspiration and injection, or two buttons, one each for aspiration and injection, respectively. Additional buttons could be added for performing other functions, e.g., to enable a Bluetooth connection to an ultrasound unit to cause it to take an ultrasound image on the screen of the ultrasound device, and share it with the base unit The ultrasound unit would have Bluetooth capability to enable such control; inclusion of such capability in the handpiece and the ultrasound unit are well within the capabilities of those of ordinary skill in the art and thus the details of same need not be included herein. Bluetooth control would take pictures as well as control the ultrasound completely (i.e. depth of ultrasound waves, gain/focus, etc). The Bluetooth component would either be disposable, sterilizable, or would include a sterilizable piece that snaps onto or is otherwise attachable to the housing.

The needle, housing, and all associated tubing and wires can be single-use (disposable). In this embodiment, the only reusable element will be the base unit.

As described herein, a foot pedal is not required for the control of the present invention.

As previously mentioned different sizes of syringes are used for injecting varying medications. Each syringe size (60 cc, 20 cc 10 cc) requires a different amount of energy added at the level of the plunger to inject the medication. Alternative embodiments of the SORADS disclosed herein include specific settings for each syringe size, thereby allowing the pump to recognize a syringe size and inject accordingly.

In an alternate embodiment two or more syringes are connected to the same needle via a stopcock or a “y-piece.” This allows two or more separate solutions to be used having different pharmacodynamic profiles. For example in one exemplary embodiment both Lidocaine and bupivacaine are to be administered. One such contemplated embodiment comprises a two-syringe pump having two separate injection tubings that meet at the level of the housing to allow for selective injection of different local anesthetics. Where the injection and aspiration are controlled by a single buttons, two separate buttons are provided that control the aspiration/injection cycles on each syringe pump individually. The tubing then combines just distal to the buttons prior to flowing out to the needle. Where the injection and aspiration are controlled by separate buttons, this embodiment requires two separate injection and two separate aspiration buttons. In other words, each of these embodiment provides a one handed means of controlling administration of multiple medicaments by way of two or more aspirating syringe pumps connected to the housing, one nerve stimulator, and tubings connecting within the housing as a “y” connector past the injection controller(s) (button(s)) and prior to the one needle. Various syringe sizes can be used with the varying medicaments.

### Exemplary Testing

For testing purposes, the SORADS device was connected to an IV bag of deionized water held at 7 feet (2.1336 meters) above the device and to a Devilbiss Vacu-Aide 721 series vacuum machine. The IV bag supplies a constant positive pressure of 157 mmHg as the injectate and the vacuum supplied a constant negative pressure of −225 mmHg to the aspirating valve. With this setup in place, each button was depressed. When the green “inject” button was depressed, fluid flowed out of the needle end. When the red “aspirate” button was depressed, fluid remaining in the needle flowed back out of the device through the aspiration tubing. Palpable suction was also noted at the end of the needle. When both buttons were depressed at the same time, fluid from the IV bag flowed directly to the aspiration valve and out to the vacuum source with no fluid delivered to the needle. When neither of the buttons were depressed, there was no fluid flow. This testing verified the proof of concept of the injecting and aspirating two button system.

The electrical component of one exemplary device was tested by building a current divider circuit in a standard breadboard and applying a constant current of 2 mA. A 1 kΩ resistor was used to mimic the resistance of human tissue. The current through the resistor was measured as the potentiometer was varied. When the resistance of the potentiometer was 10 kΩ, 2 mA of current flowed through the other resistor. As the resistance of the potentiometer was lowered, the current through the other resistor steadily decreased until it reached close to 0 mA when the potentiometer's resistance was shifted all the way to 0 kΩ. This testing verified the functionality of the current divider circuit when the resistance of the human tissue was estimated to be 1 kΩ.

Having now described a few embodiments of the invention, it should be apparent to those skilled in the art that the foregoing is merely illustrative and not limiting, having been presented by way of example only. Numerous modifications and other embodiments are within the scope of one of ordinary skill in the art and are contemplated as falling within the scope of the invention and any equivalent thereto. It can be appreciated that variations to the present invention would be readily apparent to those skilled in the art, and the present invention is intended to include those alternatives. Further, since numerous modifications will readily occur to those skilled in the art, it is not desired to limit the invention to the exact construction and operation illustrated and described, and accordingly, all suitable modifications and equivalents may be resorted to, falling within the scope of the invention.

What is claimed is:

1. A single operator anesthesia and/or drug delivery system comprising:
a nerve stimulator;
a pump;
a needle housing;
wherein the nerve stimulator is coupled to the needle housing via an electrical link, and wherein the nerve stimulator delivers an electrical nerve stimulation signal to the needle housing via the link;
wherein the pump is coupled to the needle housing via tubing;
the nerve stimulator comprising:
a nerve stimulator display; and
a nerve stimulator control;
wherein nerve stimulator control controls intensity of the nerve stimulation signal and wherein the nerve stimulator display displays intensity setting;
the pump comprising:
a pump display and
a pump control;
wherein pump control controls pump pressure and wherein the pump display displays pump setting the needle housing comprising:
a needle,
a first control; and
a second control;
wherein first control regulates intensity of the nerve stimulation signal; and
wherein the pump enables needle aspiration and drug injection, via the tubing and the needle housing.

2. The single operator anesthesia delivery system of claim 1, wherein the nerve stimulator control is a rotary or sliding control.

3. The single operator anesthesia delivery system of claim 1, wherein pump control is a valve-type control.

4. The single operator anesthesia delivery system of claim 3, wherein the pump control is an electrical control coupled to an electrically actuated valve.

5. A drug delivery system comprising:
a nerve stimulator, the nerve stimulator capable of providing electrical stimulation;
a pump, the pump capable of pumping a fluid at a pressure; and a needle housing;
wherein the nerve stimulator is coupled to the needle housing by way of an electrical link;
wherein the pump is coupled to the needle housing by way of tubing;
wherein the pump enables needle aspiration and drug delivery via the tubing and the needle housing; and
wherein the drug delivery system can be administered by one caregiver

6. The drug delivery system of claim 5, the nerve stimulator further comprising a stimulator display and a stimulator control.

7. The drug delivery system of claim 6, the pump further comprising a pump display and a pump control.

8. The drug delivery system of any of claim 7, the needle housing further comprising a needle, a first control and a second control.

9. The drug delivery system of any of claim 8, wherein intensity of a nerve stimulation signal is controllable by the stimulator control or by the first control.

10. The drug delivery system of any of claim 6, further comprising an auditory or visual signal, wherein said auditory or visual signal indicates an aspiration phase and/or an injection phase.

11. The drug delivery system of any of claim 8, wherein the pressure is controllable by the pump control or the second control; and wherein the pressure is displayable on the pump display.

12. The drug delivery system of claim 5, wherein the system is not a toggle based system.

13. The drug delivery system of claim 8, at least one of the first control or the second control further comprising a bidirectional dimmer switch, said dimmer switch having a range of outputs.

14. The drug delivery system of any preceding claim, wherein the needle housing is disposable.

15. The drug delivery system of claim 8, the first control further comprising a gate, wherein pressing the first control opens the gate allowing injection.

16. The drug delivery system of claim 8, wherein the second control is a sliding “dimmer” switch.

17. The drug delivery system of claim 5, wherein the system does not comprise a foot pedal.

18. The drug delivery system of claim 13, wherein the electrical link is a biphasic wire, and wherein said wire receives input from said dimmer switch.

19. The drug delivery system of claim 5, the pump further comprising an absolute pressure limiter, wherein said absolute pressure limiter can be set to a predetermined limit.

20. The drug delivery system of claim 5, further comprising:
an ultrasound, wherein said ultrasound provides visual feedback of the drug delivery, and wireless communication means, wherein said wireless communications means wirelessly connects the drug delivery system with the ultrasound and wherein said wireless communication means controls the ultrasound and/or transfers data from the ultrasound to the drug delivery system.
